(19) United States
(12) Patent Application Publication
(10) Pub. No.: US 2003/0093081 A1
(43) Pub. Date: May 15, 2003

(54) Interference fit screw driver

(75) Inventor: David T. Hawkes, Draper, UT (US)

Correspondence Address: KARL R CANNON, PO BOX 1909, SANDY, UT, 84091, US

(73) Assignee: Ortho Development Corporation;

(21) Appl. No.: 10/299,990

(22) Filed: Nov. 18, 2002

Related U.S. Application Data
(63) Continuation-in-part of application No. 09/894,330, filed on Jun. 28, 2001, now abandoned.

(51) Int. Cl. A61B 17/00

(52) U.S. Cl. 606/104

(57) ABSTRACT

An interference fit screw driver has a polygonal driving surface to impart torque to a corresponding surface in a screw. The end of the polygonal driving surface has a frusto-conical shaped gripping member which fits into an aperture in the screw to create a friction fit between the screw and screw driver. This allows the screw to be attached to the screw driver to facilitate installation of the screw. The friction fit is tight enough to hold the screw to the driver, yet allows the driver to be easily disengaged from the screw by pulling the driver away from the screw once the screw is lodged in place. The interference fit screw driver is particularly useful in turning bone screws into the spine of a patient during orthopedic surgery.

### CROSS-REFERENCE TO RELATED APPLICATIONS

This application is a continuation-in-part of co-pending U.S. patent application Ser. No. 09/894,330, filed Jun. 28, 2001, entitled “INTERFERENCE FIT SCREW DRIVER,” which is hereby incorporated by reference herein in its entirety, including but not limited to those portions that specifically appear hereinafter, the incorporation by reference being made with the following exception: In the event that any portion of the above-referenced application is inconsistent with this application, this application supercedes said portion of said above-referenced application.

### STATEMENT REGARDING FEDERALLY SPONSORED RESEARCH OR DEVELOPMENT

Not Applicable.

### BACKGROUND OF THE INVENTION

1. The Field of the Invention

The present invention relates generally orthopedic driving tools and fasteners, and more particularly, but not necessarily entirely, to screw drivers capable being attached to screws by an interference fit for use in orthopedic surgery, to turn bone screws into the spine of a patient.

2. Description of Related Art

A typical screw driver known in the art has a driving surface or bit which mates with a complementary recess on a screw to allow the screw driver to impart torque to the screw. An area of concern related to screw drivers has been retaining the screw on the bit of the driver while positioning the screw. This aspect of screw drivers is particularly important in the medical profession, for example, as bone screws are inserted into the spine of a patient during orthopedic surgery.

Screw drivers known in the prior art have used various techniques to attach the screws to the bits. For example, U.S. Pat. No. 4,970,922 (granted Nov. 20, 1990 to Krivec) discloses a rotatable driving tool having a plurality of substantially circularly helical driving portions with small helix angles. The driving portions mate with lobes of the fastener recess but are slightly inclined with respect to the lobes. This provides a wedge fit to retain the fastener with the tool. However, the inclined driving portions allow the driver to efficiently apply torque only in one direction. If torque is applied to the fastener in the opposite direction, the inclined driving portions force the driver out of engagement with the lobes of the fastener. Thus, a reverse driver may be required to remove the screw.

Another approach has been to use a tapered bit on the driving tool which is adapted to wedge into the recess of the screw. For example, U.S. Pat. No. 4,269,246 (granted May 26, 1981 to Larson et al.) discloses a driver bit with multiple lobes. The lobes are tapered axially, converging toward the tip end of the driver. The driver bit enters a socket of the fastener to a predetermined depth before wedging the fastener to the driver. A disadvantage of this arrangement is that the bit engages the fastener only at the outer end of the socket. This results in inefficient transfer of the torque from the driving member to the fastener. Also the concentration of force at one contact location tends to wear and deform the socket in the contact region. Furthermore, close tolerances are necessary in order to provide the proper wedge fit in a consistent manner.

U.S. Pat. No. 5,277,531 (granted Jan. 11, 1994 to Krivec) discloses another technique for attaching a fastener to a tool. This patent discloses a polygonal shaped tool fitting a socket recess formed in a fastener. The recess has planar drive surfaces alternating with sloping retaining surfaces. The polygonal tool is wedged in contact with the sloping retaining surfaces to retain the fastener on the tool. This configuration also reduces the contact area between the tool and the fastener. Furthermore, if significant torque is applied to the fastener, the retaining surfaces may become tightly wedged to the tool making it difficult to release the fastener.

An additional type of retention technique is the use of a magnetized bit on the driving tool. However, this type of retention is only useful in screws formed of magnetic material.

The prior art is thus characterized by several disadvantages that are addressed by the present invention. The present invention minimizes, and in some aspects eliminates, the above-mentioned failures, and other problems, by utilizing the methods and structural features described herein.

In view of the foregoing state of the art, it would be an advancement in the art to provide an interference fit screw driver which is simple in design and manufacture which is attached to a screw by a friction fit to facilitate positioning the screw. It would be a further advancement in the art to provide such a screw driver which is capable of applying torque in two directions and which engages the screw over a large surface area such that wear and deformation of the screw and screw driver are reduced, and which can be released from the screw even after a large torque has been applied to the screw by the screw driver. It would be an additional advancement in the art to provide a screw driver which is capable of retaining contact with screws made of nonmagnetic materials.

### BRIEF DESCRIPTION OF THE DRAWINGS

The features and advantages of the invention will become apparent from a consideration of the subsequent detailed description presented in connection with the accompanying drawings in which:

FIG. 1 is a side view of a driving tool and driven member made in accordance with the principles of the present invention, the upper portion of the driven member being illustrated in a cross-sectional view;

FIG. 1A is an enlarged, cross-sectional view of the upper portion of the driven member illustrated in FIG. 1, made in accordance with the principles of the present invention;

FIG. 2 is an end view of the driving tool of FIG. 1;

FIG. 3 is a break-away cross-sectional view of an alternative embodiment of the driving tool and driven member of FIG. 1, made in accordance with the principles of the present invention; and

FIG. 4 is an end view of the alternative embodiment of the driving member of FIG. 3.

### DETAILED DESCRIPTION OF THE INVENTION

For the purposes of promoting an understanding of the principles in accordance with the invention, reference will now be made to the embodiments illustrated in the drawings and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would normally occur to one skilled in the relevant art and having possession of this disclosure, are to be considered within the scope of the invention claimed.

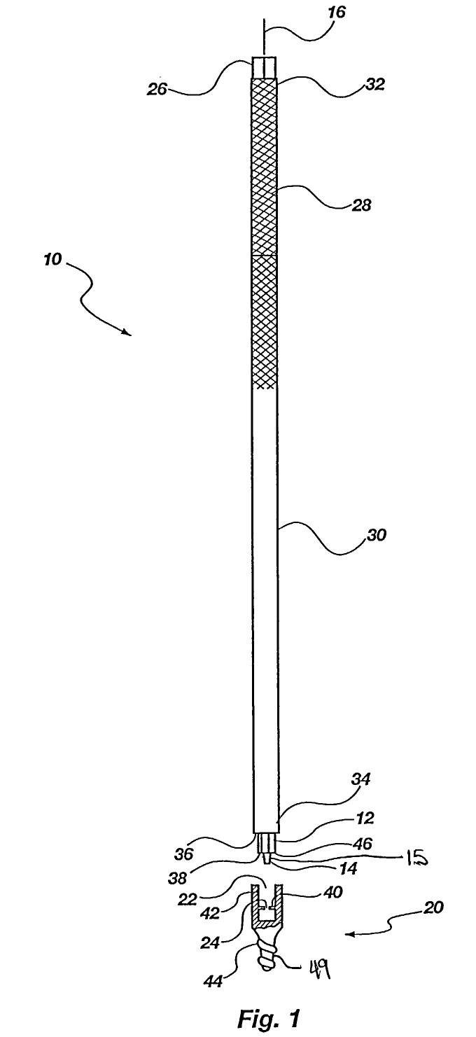

Referring now to FIG. 1, a side view is illustrated of an exemplary embodiment of a screw driver 10, also referred to herein more generally as a driving tool, and a screw 20, also referred to herein more generally as a driven member, in accordance with the principles of the present invention. The screw driver 10 is substantially cylindrical in shape having a longitudinal axis 16, and includes a body 30 having a proximal end 32 and a distal end 34. The body 30 includes a fitting 26 disposed on the proximal end 32, to allow a user to attach an implement, such as a wrench or other device for applying a force to the screw driver 10, to facilitate applying torque if necessary. The fitting 26 may be a polygonal surface or socket for example. However, as those skilled in the art will appreciate, various different configurations may be used as fittings, and such variations are intended to fall within the scope of the present invention. The body 30 may also include a handle 28 disposed on the proximal end 32 to facilitate gripping the body 30 so that torque may be applied more easily without slipping. The handle 28 may be formed of a series of grooves or knurls in the body 30 to create a frictional surface. Other varieties of handles known in the art may also be used and are intended to fall within the scope of the present invention.

A driving means 12, also referred to herein as a driving component, may be disposed on the distal end 34 of the body 30. The driving means 12 has a first end 36, a second end 38, and may comprise a constant cross-sectional configuration throughout the length of the driving means 12. The constant cross-section in that example allows efficient transfer of torque from the screw driver 10 to the screw 20 without the tendency to separate as is common with tapered driving surfaces. Also, such constant cross-sectional configuration allows the driving means 12 to impart torque efficiently when rotated in both clockwise and counter-clockwise directions. This feature is an improvement over driving surfaces with curved or angled driving surfaces, which work to transfer torque efficiently in only one direction.

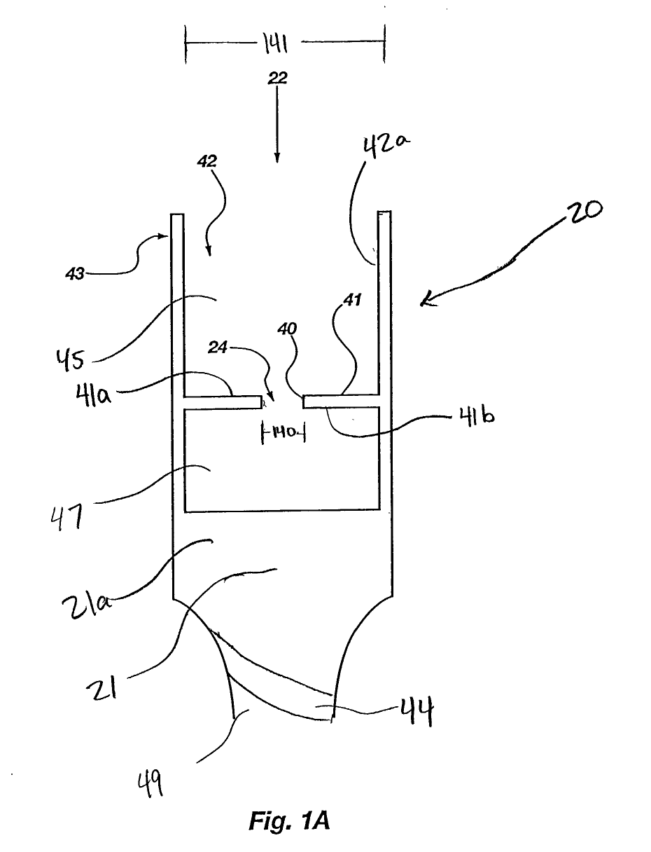

The driving means 12 may be polygonal in shape. For example, the polygonal shape of the driving means 12 may be a hexagonal shape as shown most clearly in FIG. 2. However, as those skilled in the art will appreciate, driving surfaces of various shapes, such as star shapes, cross shapes, blade shapes, or fluted configurations, may be used for the shape of the driving means 12 and such shapes are intended to fall within the scope of the present invention.

A gripping means 14, also referred to herein as a gripping surface 14, may be disposed on the second end 38 of the driving means 12 such that the gripping means extends below said second end 38 for gripping the screw 20 and to removably attach the screw 20 to the screw driver 10. It will be appreciated that the gripping means 14 may be substantially frusto-conical in shape, however other configurations of the gripping means 14 are possible, and are intended to fall within the scope of the present invention. The gripping means 14 may be located along the axis 16 such that a break 46 may be disposed radially from the longitudinal axis 16 between the driving means 12 and the gripping means 14. The separation of the gripping means 14 from the driving means 12 allows the gripping means 14 to function independently from the driving means 12 and vice versa. The function of the gripping means 14 will be described more fully below.

As illustrated in FIG. 2, the driving means 12 may have a radial dimension 48 from the longitudinal axis 16 of the tool 10 which may be larger than a radial dimension 50 of the gripping means 14. The difference in radial dimension forms the radial break 46 to separate the drive means 12 from the gripping means 14, and the difference in radial dimension provides a larger surface area to the driving means 12 for efficiently applying torque rather than for gripping the screw 20. The radial dimension 48 of the driving means 12 may be sized in a range of between approximately two to four times the radial dimension 50 of the gripping means 14 from the longitudinal axis 16.

As illustrated in FIGS. 1 and 1A, the screw 20 may include a first aperture 24 and a second aperture 22. The first aperture 24 may be aligned coaxially with the second aperture 22 and may be smaller than the second aperture 22, such that the second aperture 22 circumscribes the first aperture 24. The first aperture 24 may be defined by a circumferential edge 40, which circumferential edge 40 may be engaged by a portion of an outer surface of the gripping means 14 to attach the screw 20 to the screw driver 10. The first aperture 24 and the circumferential edge 40 are illustrated as being circular in shape, however, various shapes may be used for the first aperture 24 and the circumferential edge 40, and each of the various shapes are intended to fall within the scope of the present invention. As further illustrated in FIGS. 1 and 1A, the circumferential edge 40 defines a diameter 140 of the first aperture 24 that may be less than twenty percent of a length of a diameter 141 of the second aperture 22. More specifically, the diameter of the first aperture 24 may be less than three millimeters in length.

The second aperture 22 may be defined by a socket 42, sometimes referred to herein as a receiving cavity, which comprises a surface 42a that engages the driving means 12 of the screw driver 10 to form an interference fit. The socket 42 may have a shape which corresponds to the shape of the driving means 12, such as a hexagonal shape for example. However, it will be appreciated that the socket 42 may have various corresponding shapes of polygons, stars, crosses, blades, or fluted configurations for example, that are intended to fall within the scope of the present invention.

The screw 20 may also have an engaging means for advancing the driven member into the patient's bone, said engaging means comprising a shank 49 with threads 44 of any variety known in the art located thereon. However, the principles of the present invention may be applied to any such driven member 20 which may employ other engaging means for advancing the driven member into the patient's bone such as flanges or pins for example, in addition to threads 44.

In use, the screw 20 may be attached to the screw driver 10 by inserting the gripping means 14 into the first aperture 24 to the point where the edge 40 engages the gripping means 14 to wedge the gripping means 14 against the edge 40 with a friction fit. The area of contact between the gripping means 14 and the edge 40 may be large enough to supply sufficient force to attach the screw 20 to the screw driver 10, yet small enough such that the screw 20 may be released when desired without undue effort. As the gripping means 14 enters the first aperture 24, the driving means 12 may be aligned against the socket 42. The surface area of the contact between the driving means 12 and the socket 42 may be large as compared to the contact between the gripping means 14 and the edge 40. This relationship allows efficient transfer of torque from the screw driver 10 to the screw 20 without imposing concentrated loads on a single point. Furthermore, since the contact between the driving means 12 and the socket 42 may be separate from the contact between the gripping means 14 and the edge 40, the screw 20 can be easily released from the screw driver 10, regardless of how much torque is applied. In other words, a high torque placed on the socket 42 by the driving means 12, may have no effect on the frictional connection between the gripping means 14 and the edge 40. Once it is desired to release the screw 20 from the screw driver 10, the screw driver 10 may be simply pulled from the screw 20 with a force sufficient to overcome the frictional fit between the gripping means 14 and the edge 40.

It will be appreciated that the friction fit of the present invention occurs between a fractional portion of the outer surface of the gripping means 14 and the circumferential edge 40, which friction fit occurs along a portion of the gripping means 14 and the circumferential edge 40 that is substantially less than a majority. Therefore, it will be appreciated that the phrase “fractional engagement” shall refer to the concept that a gripping piece 41 engages with a some fractional portion of the surface of another member in which said fractional portion is substantially less than a majority of its surface. One embodiment of this concept of “fractional engagement” is illustrated and characterized by the absence of full contact along a majority of the surface of the gripping means 14 and the circumferential edge 40 of the driven member 20. Additionally, the friction fit of the present invention is not a press-fit, and the gripping means 14 does not bite into, or otherwise deform, the circumferential edge or any other portion of the driven member 20. As used herein, the term “bite” refers to the slight deformation that occurs in the driven member as an end of the screw driver is located within said driven member.

Referring specifically to FIG. 1A, which is an enlarged view of the screw 20 of FIG. 1, socket 42 may be defined by a sidewall 43, which extends upwardly from a base 21a of a head portion 21 of the screw 20. An annular gripping piece 41 may extend radially inward from sidewall 43 of the socket 42 of the screw 20 and further provides the circumferential edge 40, which frictionally engages a portion of gripping means 14. It will be appreciated that the gripping piece 41 essentially separates the socket 42, or receiving cavity, into a first chamber 45 and a second chamber 47. Also illustrated in FIG. 1A is the first aperture 24 and the second aperture 22, wherein the second aperture 22 may be circumscribed by sidewall 43 and may have the gripping piece 41 located within the socket 42 as illustrated. The gripping piece 41 comprises an upper surface 41a and a lower surface 41b, wherein the upper surface 41a faces the first chamber 45 and the lower surface 41b faces the second chamber 47. It will be appreciated that the two chambers 45 and 47 may not be completely enclosed chambers, although such a configuration is contemplated by the present invention, but the chambers 45 and 47 may both be essentially part of the socket 42, and may be separated by the gripping piece 41.

Manufacturing of the present invention may be facilitated since the gripping means 14 and edge 40 need not be constructed to exact dimensions to allow proper attachment of the screw 20 to the screw driver 10. The gripping means 14 comprises a tapered surface 15 that allows the friction fit to occur between the tapered surface 15 and the edge 40 at various longitudinal locations along the gripping means 14 depending upon the size of the first aperture 24 in relation to the taper of the gripping means 14. Furthermore, since the screw 20 may be attached to the screw driver 10 by the friction fit, the use of magnetic materials is not necessary. The screw driver 10 and screw 20 may be constructed of various biocompatible materials known to those skilled in the art.

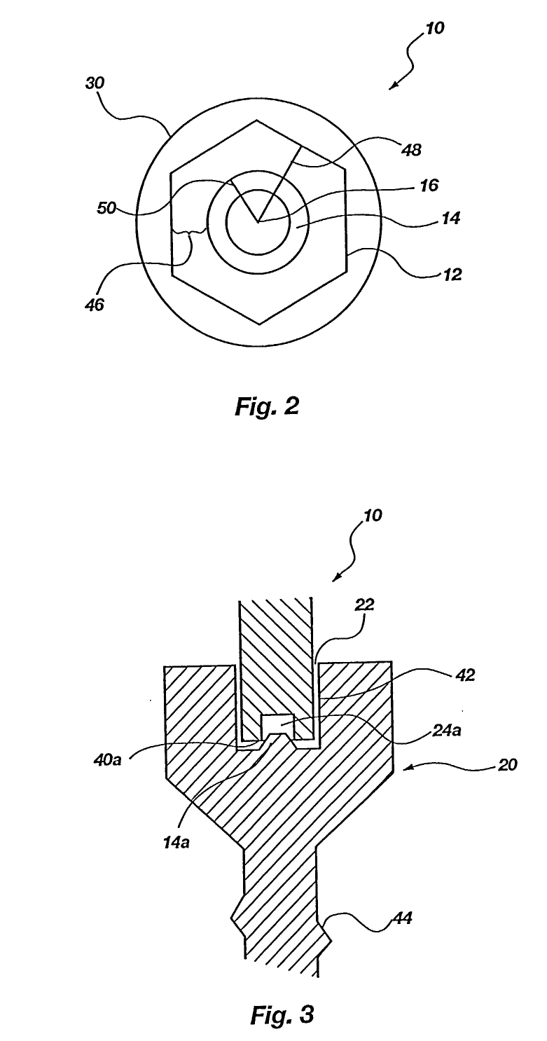

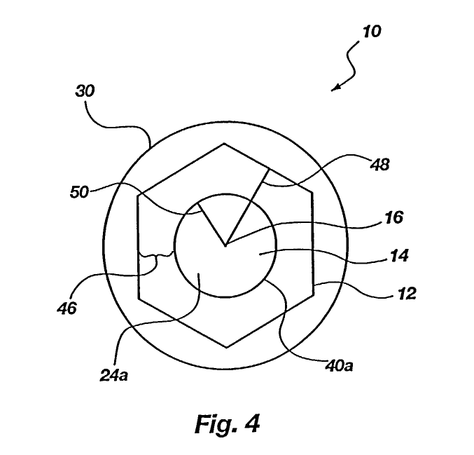

Reference will now to made to FIG. 3 to describe a second embodiment of the present invention. As previously discussed, the present embodiments of the invention illustrated herein are merely exemplary of the possible embodiments of the invention, including that illustrated in FIG. 3.

It will be appreciated that the second embodiment of the invention illustrated in FIG. 3 contains many of the same structures represented in FIGS. 1-2 and only the new or different structures will be explained to most succinctly explain the additional advantages which come with the embodiments of the invention illustrated in FIG. 3. The second embodiment of the invention includes a tapered member 14a disposed on the screw 20, and a gripping means including a first aperture 24a and edge 40a disposed on the screw driver 10. The function of the second embodiment of the invention is similar to that of the first embodiment. An advantage of the second embodiment is that the tapered member 14a is protected within the socket 42. Therefore, damage to the tapered member 14a is less likely so that a proper fit between the screw 20 and the screw driver 10 may be allowed.

In accordance with the features and combinations described above, a useful method of driving a driven member 20 with a tool 10 includes the steps of:

A) inserting a gripping means 14 into a first aperture 24 to attach the driven member 20 to the tool 10; and

B) inserting a driving means 12 into a second aperture 22 to transfer a driving force from the tool 10 to the driven member 20.

In view of the foregoing, it will be appreciated that the present invention provides an interference fit screw driver which is simple in design and manufacture which is attached to a screw by a friction fit to facilitate positioning the screw. The present invention also provides such a screw driver which is capable of applying torque in two directions and which engages the screw over a large surface area such that wear and deformation of the screw and screw driver are reduced. The present invention also provides a screw driver which can be released from the screw even after a large torque has been applied to the screw by the screw driver. The present invention also provides a screw driver which is capable of retaining contact with screws made of nonmagnetic materials.

Those having ordinary skill in the relevant art will appreciate the advantages provided by the potential features of the present invention. For example, it is a potential feature of the present invention to provide an interference fit screw driver which is simple in design and manufacture. It is another potential feature of the present invention to provide a screw driver which is capable of driving a screw by way of an interference fit. It is a further potential feature of the present invention to provide an interference fit screw driver which is attached to a screw by a friction fit to facilitate positioning the screw during a surgical procedure. It is another potential feature of the present invention to provide such a screw driver which is capable of applying torque in two directions. It is a further potential feature of the present invention, in accordance with one aspect thereof, to provide a screw driver which engages the screw over a large surface area such that wear and deformation of the screw and screw driver are reduced.

It is an additional potential feature of the invention, in accordance with one aspect thereof, to provide an interference fit screw driver which can be released from the screw even after a large torque has been applied to the screw by the screw driver. It is another potential feature of the present invention to provide a screw driver which is capable of retaining contact with screws made of nonmagnetic materials.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. An orthopedic device for use during orthopedic surgery, the device comprising:
a driven member configured to be driven into a patient's bone, the driven member comprising:
a sidewall defining and circumscribing a receiving cavity;
a gripping piece extending radially inward from the sidewall separating the receiving cavity, said gripping piece having an upper surface, a lower surface and a circumferential edge defining a first aperture; and
wherein said sidewall further defines a second aperture leading into the receiving cavity;
a driving tool configured for driving the driven member into the patient's bone, the driving tool comprising:
a body having a proximal end and a distal end;
a driving means disposed on the distal end of the body for engaging a surface of the receiving cavity and contactibly driving said driven member; and
a gripping means disposed on a distal end of the driving means for engaging in a grip with the gripping piece such that a fractional engagement between said gripping piece and said gripping means occurs, whereby only a fractional portion of said gripping means is disposed in contact with said circumferential edge of the gripping piece in a friction fit.

2. The orthopedic device of claim 1, wherein the gripping piece separates the receiving cavity into a first chamber and a second chamber.

3. The orthopedic device of claim 2, wherein the upper surface of the gripping piece faces toward the first chamber and the lower surface of the gripping piece faces toward the second chamber.

4. The orthopedic device of claim 1, wherein the first aperture has a diameter that is less than twenty percent of a length of a diameter of the second aperture.

5. The orthopedic device of claim 4, wherein the diameter of first aperture is less than three millimeters in length.

6. The orthopedic device of claim 1, wherein the first aperture is coaxially aligned with the second aperture.

7. The orthopedic device of claim 1, wherein the first aperture is smaller than the second aperture, such that the second aperture circumscribes the first aperture.

8. The orthopedic device of claim 1, wherein the first aperture and the circumferential edge are circular in shape.

9. The orthopedic device of claim 1, wherein the driven member further comprises an engaging means for advancing the driven member into the patient's bone.

10. The orthopedic device of claim 9, wherein the engaging means comprises a shank having threads located thereon.

11. The orthopedic device of claim 1, wherein the engagement between the surface of the receiving cavity and the driving means forms an interference fit such that when torque is applied by a user the surface area of contact between the driving means and the receiving cavity is large, as compared to the contact between the gripping means and the circumferential edge, which allows efficient transfer of torque from the driving tool to the driven member without imposing concentrated loads on a single point.

12. The orthopedic device of claim 1, wherein the engagement between the driving means and the receiving cavity is separate and distinct from the fractional engagement between the gripping means and the circumferential edge, such that the driven member can be easily released from the driving tool, regardless of how much torque is applied to said driving tool and said driven member.

13. The orthopedic device of claim 1, wherein the gripping means comprises a tapered outer surface such that fractional engagement between the gripping means and the circumferential edge is characterized by the absence of full contact along a majority of the tapered outer surface of the gripping means and the circumferential edge of the driven member.

14. The orthopedic device of claim 1, wherein said driving means has a polygonal cross section.

15. The orthopedic device of claim 14, wherein said driving means has a hexagonal cross section.

16. The orthopedic device of claim 1, wherein said driving means has a constant cross sectional configuration.

17. The orthopedic device of claim 1, wherein said driving tool further comprises a fitting that is disposed on said proximal end of said body to facilitate imparting torque to the driving tool.

18. The orthopedic device of claim 1, wherein said driving tool comprises a handle that is disposed on the proximal end of the body to facilitate imparting torque to the driving tool.

19. The orthopedic device of claim 1, wherein said body has a substantially cylindrical shaped configuration.

20. The orthopedic device of claim 1, wherein said gripping means has a smaller surface area than said driving means.

21. The orthopedic device of claim 1, wherein said gripping means has a tapered configuration.

22. The orthopedic device of claim 1, wherein said gripping means comprises a substantial frusto-conical shape.

23. The orthopedic device of claim 1, wherein said driving means has a radial dimension from a longitudinal axis of said driving tool which is sized in a range of between approximately two to four times a radial dimension of said gripping means from said longitudinal axis.

24. The orthopedic device of claim 1, wherein said body has a radial dimension which is greater than a radial dimension of said driving means, and wherein the radial dimension of said driving means is greater than a radial dimension of said gripping means.

25. The orthopedic device of claim 1, wherein the gripping piece resides between the gripping means and the sidewall defining the receiving cavity such that said gripping means is held spaced apart from and out of contact with said sidewall.

26. An orthopedic device and tool for use during orthopedic surgery, the device comprising:
a driven member configured to be driven into a patient's bone, the driven member comprising:
a sidewall defining and circumscribing a receiving cavity;
a gripping piece extending radially inward from the sidewall separating the receiving cavity, said gripping piece having an upper surface, a lower surface and a circumferential edge defining a first aperture; and
wherein said sidewall further defines a second aperture leading into the receiving cavity;
a driving tool configured for driving the driven member into the patient's bone, the driving tool comprising:
a body having a proximal end and a distal end;
a driving component having a substantially constant cross section disposed on the distal end of the body, said driving component being configured and dimensioned for engaging a surface of the receiving cavity and contactibly driving said driven member; and
a tapered surface disposed on a distal end of the driving component for engaging in a grip with the gripping piece such that a fractional engagement between said gripping piece and said tapered surface occurs, whereby only a fractional portion of said tapered surface is disposed in contact with said circumferential edge of the gripping piece in a friction fit.

27. The orthopedic device and tool of claim 26, wherein the gripping piece separates the receiving cavity into a first chamber and a second chamber.

28. The orthopedic device and tool of claim 27, wherein the upper surface of the gripping piece faces toward the first chamber and the lower surface of the gripping piece faces toward the second chamber.

29. The orthopedic device and tool of claim 26, wherein the first aperture has a diameter that is less than twenty percent of a length of a diameter of the second aperture.

30. The orthopedic device and tool of claim 29, wherein the diameter of first aperture is less than three millimeters in length.

31. The orthopedic device and tool of claim 26, wherein the first aperture is coaxially aligned with the second aperture.

32. The orthopedic device and tool of claim 26, wherein the first aperture is smaller than the second aperture, such that the second aperture circumscribes the first aperture.

33. The orthopedic device and tool of claim 26, wherein the first aperture and the circumferential edge are circular in shape.

34. The orthopedic device and tool of claim 26, wherein the driven member further comprises an engaging means for advancing the driven member into the patient's bone.

35. The orthopedic device and tool of claim 34, wherein the engaging means comprises a shank having threads located thereon.

36. The orthopedic device and tool of claim 26, wherein the engagement between the surface of the receiving cavity and the driving component forms an interference fit such that when torque is applied by a user the surface area of contact between the driving component and the receiving cavity is large, as compared to the contact between the tapered surface and the circumferential edge, which allows efficient transfer of torque from the driving tool to the driven member without imposing concentrated loads on a single point.

37. The orthopedic device and tool of claim 26, wherein the engagement between the driving component and the receiving cavity is separate and distinct from the fractional engagement between the tapered surface and the circumferential edge, such that the driven member can be easily released from the driving tool, regardless of how much torque is applied to said driving tool and said driven member.

38. The orthopedic device and tool of claim 26, wherein the fractional engagement between the tapered surface and the circumferential edge is characterized by the absence of full contact along a majority of the tapered surface and the circumferential edge of the driven member.

39. The orthopedic device and tool of claim 26, wherein said driving component has a polygonal cross section.

40. The orthopedic device and tool of claim 39, wherein said driving component has a hexagonal cross section.

41. The orthopedic device and tool of claim 26, wherein said driving tool further comprises a fitting that is disposed on said proximal end of said body to facilitate imparting torque to the driving tool.

42. The orthopedic device and tool of claim 26, wherein said driving tool comprises a handle that is disposed on the proximal end of the body to facilitate imparting torque to the driving tool.

43. The orthopedic device and tool of claim 26, wherein said body has a substantially cylindrical shaped configuration.

44. The orthopedic device and tool of claim 26, wherein said tapered surface has a smaller surface area than said driving component.

45. The orthopedic device and tool of claim 26, wherein said tapered surface comprises a substantial frusto-conical shape.

46. The orthopedic device and tool of claim 26, wherein said driving component has a radial dimension from a longitudinal axis of said driving tool which is sized in a range of between approximately two to four times a radial dimension of said tapered surface from said longitudinal axis.

47. The orthopedic device and tool of claim 26, wherein said body has a radial dimension which is greater than a radial dimension of said driving component, and wherein the radial dimension of said driving component is greater than a radial dimension of said tapered surface.

48. An orthopedic device and tool for use during orthopedic surgery, the device comprising:
a driven member configured to be driven into a patient's bone, the driven member comprising:
a sidewall defining and circumscribing a receiving cavity; and
a gripping piece extending radially inward from the sidewall;
a driving tool configured for driving the driven member into the patient's bone, the driving tool comprising:
a body having a proximal end and a distal end;
a driving means disposed on the distal end of the body for engaging a surface of the receiving cavity and contactibly driving said driven member; and
a gripping surface disposed on a distal end of the driving means for engaging in a grip with the gripping piece such that a fractional engagement between said gripping piece and said gripping surface occurs, whereby only a fractional portion of said gripping surface is disposed in contact with said circumferential edge of the gripping piece in a friction fit.

49. The orthopedic device and tool of claim 48, wherein said gripping piece is annular and further comprises an upper surface, a lower surface and a circumferential edge defining a first aperture.

50. The orthopedic device and tool of claim 49, wherein said sidewall further defines a second aperture leading into the receiving cavity.

51. The orthopedic device and tool of claim 49, wherein said gripping piece essentially separates the receiving cavity into at least a first chamber and a second chamber.

52. The orthopedic device and tool of claim 51, wherein the upper surface of the gripping piece faces toward the first chamber and the lower surface of the gripping piece faces toward the second chamber.

53. The orthopedic device and tool of claim 50, wherein the first aperture has a diameter that is less than twenty percent of a length of a diameter of the second aperture.

54. The orthopedic device and tool of claim 53, wherein the diameter of first aperture is less than three millimeters in length.

55. The orthopedic device and tool of claim 50, wherein the first aperture is coaxially aligned with the second aperture.

56. The orthopedic device and tool of claim 50, wherein the first aperture is smaller than the second aperture, such that the second aperture circumscribes the first aperture.

57. The orthopedic device and tool of claim 49, wherein the first aperture and the circumferential edge are circular in shape.

58. The orthopedic device and tool of claim 48, wherein the driven member further comprises an engaging means for advancing the driven member into the patient's bone.

59. The orthopedic device and tool of claim 58, wherein the engaging means comprises a shank having threads located thereon.

60. The orthopedic device and tool of claim 48, wherein the engagement between the surface of the receiving cavity and the driving means forms an interference fit such that when torque is applied by a user the surface area of contact between the driving means and the receiving cavity is large, as compared to the contact between the gripping surface and the gripping piece, which allows efficient transfer of torque from the driving tool to the driven member without imposing concentrated loads on a single point.

61. The orthopedic device and tool of claim 48, wherein the engagement between the driving means and the receiving cavity is separate and distinct from the fractional engagement between the gripping surface and the gripping piece, such that the driven member can be easily released from the driving tool, regardless of how much torque is applied to said driving tool and said driven member.

62. The orthopedic device and tool of claim 48, wherein the gripping surface comprises a tapered outer surface such that fractional engagement between the gripping surface and the gripping piece is characterized by the absence of full contact along a majority of the tapered outer surface of the gripping surface and the gripping piece of the driven member.

63. The orthopedic device and tool of claim 48, wherein said driving means has a polygonal cross section.

64. The orthopedic device and tool of claim 63, wherein said driving means has a hexagonal cross section.

65. The orthopedic device and tool of claim 48, wherein said driving means has a constant cross sectional configuration.

66. The orthopedic device and tool of claim 48, wherein said driving tool further comprises a fitting that is disposed on said proximal end of said body to facilitate imparting torque to the driving tool.

67. The orthopedic device and tool of claim 48, wherein said driving tool comprises a handle that is disposed on the proximal end of the body to facilitate imparting torque to the driving tool.

68. The orthopedic device and tool of claim 48, wherein said body has a substantially cylindrical shaped configuration.

69. The orthopedic device and tool of claim 48, wherein said gripping surface has a smaller surface area than said driving means.

70. The orthopedic device and tool of claim 48, wherein said gripping surface has a tapered configuration.

71. The orthopedic device and tool of claim 48, wherein said gripping surface comprises a substantial frusto-conical shape.

72. The orthopedic device and tool of claim 48, wherein said driving means has a radial dimension from a longitudinal axis of said driving tool which is sized in a range of between approximately two to four times a radial dimension of said gripping means from said longitudinal axis.

73. The orthopedic device and tool of claim 48, wherein said body has a radial dimension which is greater than a radial dimension of said driving means, and wherein the radial dimension of said driving means is greater than a radial dimension of said gripping surface.

74. A combination orthopedic device comprising:
a driven member having a sidewall and a gripping piece extending radially inward from the sidewall, wherein the driven member further includes a second aperture defined by the sidewall, and a first aperture defined by the gripping piece; and
a driving tool comprising a driving means and a tapered surface;
wherein the tapered surface of the driving tool engages said gripping piece in a grip such that said gripping piece engages the tapered surface in fractional engagement to thereby attach said driven member to said driving tool when said driving means resides in contact with the sidewall of said second aperture to thereby impart a driving force to said driven member.

75. An orthopedic device and tool for use during orthopedic surgery, the device comprising:
a driven member configured to be driven into a patient's bone, the driven member comprising:
a sidewall defining and circumscribing a receiving cavity;
a gripping piece extending radially inward from the sidewall separating the receiving cavity, said gripping piece having an upper surface, a lower surface and a circumferential edge defining a first aperture; and
a driving tool configured for driving the driven member into the patient's bone, the driving tool comprising a driving component for engaging a surface of the receiving cavity to thereby exert a driving force on said driven member, and a gripping surface engaging said circumferential edge in a grip such that said gripping piece engages the gripping surface in fractional engagement thereby attaching said driving tool to said driven member by way of a friction fit.

76. An orthopedic device and tool for use during orthopedic surgery, the device comprising:
a driven member configured to be driven into a patient's bone, the driven member comprising:
a sidewall defining and circumscribing a receiving cavity; and
a gripping piece extending radially inward from the sidewall substantially separating the receiving cavity into at least a first chamber and a second chamber;
a driving tool configured for driving the driven member into the patient's bone, the driving tool comprising a driving component for engaging a surface of the receiving cavity and for driving said driven member, and a gripping surface for engaging said driven member in a grip such that at least a portion of the gripping piece fractionally engages a portion of said gripping surface in a friction fit.

77. The orthopedic device and tool of claim 76, wherein said gripping piece is annular and comprises an upper surface, a lower surface and a circumferential edge defining a first aperture.

78. The orthopedic device and tool of claim 76, wherein said sidewall further defines a second aperture leading into the receiving cavity.

79. A method of attaching a driving tool to a driven member, including the steps of:
providing a sidewall defining a receiving cavity, and a gripping piece that extends radially inward from said sidewall that defines a first aperture, wherein said sidewall further defines a second aperture on the driven member;
providing a driving component on a distal end of the driving tool, and a gripping surface extending from said driving component on the driving tool;
inserting the gripping surface into the first aperture such that a portion of the gripping surface engages a portion of the gripping piece in a grip such that the gripping piece fractionally engages said gripping surface, whereby only a fractional portion of said gripping surface is disposed in contact with said gripping piece in a friction fit; and
inserting the driving component into the second aperture simultaneously with the step of inserting the gripping surface into the first aperture, such that the driving component engages the sidewall to thereby transfer a driving force from the driving tool to the driven member.
